(19) United States
(12) Patent Application Publication
(10) Pub. No.: US 2012/0123418 A1
(43) Pub. Date: May 17, 2012

(54) STABILIZERS FOR SURGICAL TOOLS

(75) Inventors: Marius Giurgi, Pittsburgh, PA (US); Adam Hahn, Pittsburgh, PA (US); Benjamin McCandless, Pittsburgh, PA (US); Constantinos Nikou, Monroeville, PA (US)

(73) Assignee: Blue Belt Technologies, LLC, Pittsburgh, PA (US)

(21) Appl. No.: 13/296,882

(22) Filed: Nov. 15, 2011

Related U.S. Application Data
(60) Provisional application No. 61/414,196, filed on Nov. 16, 2010.

(51) Int. Cl. A61B 17/17; A61B 17/15

(52) U.S. Cl. 606/80; 606/82

(57) ABSTRACT

Described herein are stabilizers for surgical tools. One aspect provides a surgical tool stabilizer, comprising: a support configured to engage at least a portion of a surgical tool and configured to receive at least a portion of a tracking system; and a retractable stabilizer configured to surround at least a portion of an end effector of said surgical tool. Other embodiments are described.

### CLAIM FOR PRIORITY

This application claims priority to U.S. Provisional Application Ser. No. 61/414,196, filed on Nov. 16, 2010, which is incorporated by reference herein.

### BACKGROUND

Surgical saws and drills are among the variety of surgical tools included in a surgeon's armamentarium. Surgical saws and drills play a role in a large number of procedures each year. For example, in bone surgery, such as for example in a total knee replacement (TKR) procedure, it is important to prepare a patient's bone to accept an implant in an anatomically correct, precise location. With approximately 581,000 knee replacements performed each year in the United States, even a small percentage of those replacements manifesting substandard fit of the implants may lead to significantly expensive “re-fitting” procedures involving additional surgical time and effort.

Accurate osteotomy drilling/cuts are thus essential to most orthopaedic surgical procedures. Navigated surgical tools may be used to enhance accuracy. A navigated surgical tool may use electronic navigation to locate, fixate, adjust and/or correct the trajectory and cutting rate of a cutting tool based on a user-defined surgical plan, while allowing a surgeon to use a freehand cutting motion. Navigated surgical tools may include elements such as a cutting drill having a rotating bur end effector that provides cuts on the bone, or a saw with a blade end effector that is used to make planar cuts in bone.

In order to assist in the cutting, a user (surgeon) may perform surgery using a navigation system for additional guidance and understanding of the location of the tool being used. Other systems may be employed to fully automate the cutting, for example in the case of a robotic navigation system having a fixed target to assure a pre-determined cut. Example navigation systems exist that provide a surgeon with control by determining a distance between a cutting tool being used and a target shape and assist the surgeon in making the desired shape (on the target/bone).

### BRIEF SUMMARY

In summary, one aspect provides a surgical tool stabilizer, comprising: a support configured to engage at least a portion of a surgical tool and configured to receive at least a portion of a tracking system; and a retractable stabilizer configured to surround at least a portion of an end effector of said surgical tool.

Another aspect provides a surgical tool stabilizer, comprising: a support configured to engage at least a portion of a surgical saw and configured to receive at least a portion of a tracking system; and a retractable stabilizer configured to surround at least a portion of an end effector of said surgical saw; wherein said retractable stabilizer further comprises: a guided slot portion having a slot defined therein; wherein said slot is configured to enclose and stabilize said end effector.

A further aspect provides a surgical tool stabilizer, comprising: a support configured to engage at least a portion of a surgical drill and configured to receive at least a portion of a tracking system; and a retractable stabilizer configured to surround at least a portion of an end effector of said surgical drill; wherein said retractable stabilizer further comprises a cannulated, plungable drill stabilizer.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example surgical environment.

FIG. 2 illustrates an example surgical saw.

FIG. 3(A-B) illustrates example locations for bone cuts and implant placement in an example surgical procedure.

FIG. 4 illustrates an example surgical saw and stabilizer.

FIG. 5 illustrates a perspective view of a portion of an example surgical saw stabilizer.

FIG. 6 illustrates a top view of a portion of an example surgical saw stabilizer.

FIG. 7 illustrates a perspective view of a portion of an example surgical saw and stabilizer.

FIG. 8 illustrates a perspective view of a portion of an example surgical saw and stabilizer.

FIG. 9 illustrates an enlarged view of a portion of an example surgical saw and stabilizer.

FIG. 10 illustrates a side view of a portion of an example surgical saw and stabilizer.

FIG. 11 illustrates an example surgical environment.

FIG. 12 illustrates an example surgical drill hand piece and stabilizer.

FIG. 13 illustrates an enlarged view of an example surgical drill hand piece and stabilizer.

FIG. 14 illustrates an exploded view of an example stabilizer for a surgical drill.

FIG. 15 illustrates an end view of an example stabilizer for a surgical drill.

FIG. 16 illustrates a side view of an example stabilizer for a surgical drill.

FIG. 17 illustrates a side view of an example stabilizer for a surgical drill.

FIG. 18 illustrates a perspective view of an example stabilizer for a surgical drill.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the claims, but is merely representative of those embodiments.

Reference throughout this specification to “embodiment(s)” (or the like) means that a particular feature, structure, or characteristic described in connection with the embodiment is included in at least one embodiment. Thus, appearances of the phrases “according to embodiments” or “an embodiment” (or the like) in various places throughout this specification are not necessarily all referring to the same embodiment.

Furthermore, the described features, structures, or characteristics may be combined in any suitable manner in different embodiments. In the following description, numerous specific details are provided to give a thorough understanding of example embodiments. One skilled in the relevant art will recognize, however, that aspects can be practiced without certain specific details, or with other methods, components, materials, et cetera. In other instances, well-known structures, materials, or operations are not shown or described in detail to avoid obfuscation.

Certain surgical procedures, for example, total knee replacement (TKR) procedures, typically require a number of planar cuts to be made with an oscillating saw in order to prepare the bones for placement of implants. A tool such as a navigated sagittal saw may be used for such surgical procedures. As is known, surgical navigation tracks markers in space to extrapolate the position of an instrument (for example, a sagittal saw) that the markers are rigidly attached to. However, if there is any flex or deviation of the rigid body from the relative tracker position, there will be error introduced in the tracking system. Such may be the case when navigating a tracked sagittal saw while performing a cutting procedure like that involved in a TKR.

During a cut, the surgeon will apply forces (intentional and unintentional) and torques to the saw. The saw blade is necessarily thin (and therefore flexible), and the interface that locks the blade to the saw may have significant play in it. Furthermore, the markers are typically rigidly attached to the saw body. These forces deflect the oscillating saw blade with respect to the markers and may introduce error in the navigation tracking system. Such errors may result in cuts that are over or under the target cutting surface and lead to substandard fit of the implant on the bone.

Therefore, it is desirable to devise an apparatus that can be used with a surgical saw and can provide means to decrease flexure of the saw blade during the bone cutting procedure (including the initial cut), for example as may be used in a TKR surgery, so as to increase cut accuracy. It is further desirable that the apparatus be able to facilitate careful preparation of a patient's bone to accept an implant in an anatomically correct and precise location, without significantly adding to the cost or duration of the surgical procedure. Such an apparatus should also provide a stable interface with the bone on which a surgeon can adjust cut trajectory using surgical navigation.

Accordingly, an embodiment provides a blade stabilizer for a surgical saw that may use optical navigation to locate and adjust the trajectory of the cutting plane based on a user-defined surgical plan, while allowing the surgeon to use a freehand cutting motion. An embodiment provides a retractable blade stabilizer that decreases flexure of the saw blade during a cutting procedure, while allowing the blade to cut freely.

Similarly, surgical drill stability is required in various surgical procedures. Walking or slippage of the drill bit (cutting/effector end) at the point of contact may cause inaccurate cutting. This lack of control my reduce accuracy of the hole/cutting produced, increase damage, and reduce the overall quality of the procedure.

Accordingly, an embodiment provides a drill stabilizer that guides and stabilizes a surgical drill bit. An embodiment provides a drill stabilizer that is cannulated, plungable and includes a fixing means, such as a plurality of teeth/spikes, that keeps a distal end of the drill stabilizer adhered to bone in a stable positioning with respect to the point of contact. This facilitates drill bit entry into the bone without walking or slippage.

The description now turns to the figures. The illustrated example embodiments will be best understood by reference to the figures. The following description is intended only by way of example and simply illustrates certain example embodiments representative of the invention, as claimed.

As described herein, surgical procedures such as a TKR typically require a number of planar cuts to be made in order to prepare the bones for placement of implants (see for example FIG. 3(A-B)). It is widely accepted that one of the main goals of TKR reconstruction surgery is restoration of the mechanical axis of the knee. This means that the axis connecting the centers of the hip and knee on the femur, and the axis connecting the centers of the knee and ankle on the tibia should be collinear after surgery. Alignment within three degrees is considered acceptable and larger discrepancies are considered to lead to the early failure of the joint replacement. Conventional TKR procedures rely on the surgeon's “eyeballed” estimate of the centers of the hip and ankle, or on the measured or estimated angles between the femoral medullary canal and the mechanical axis, which are not always reliable.

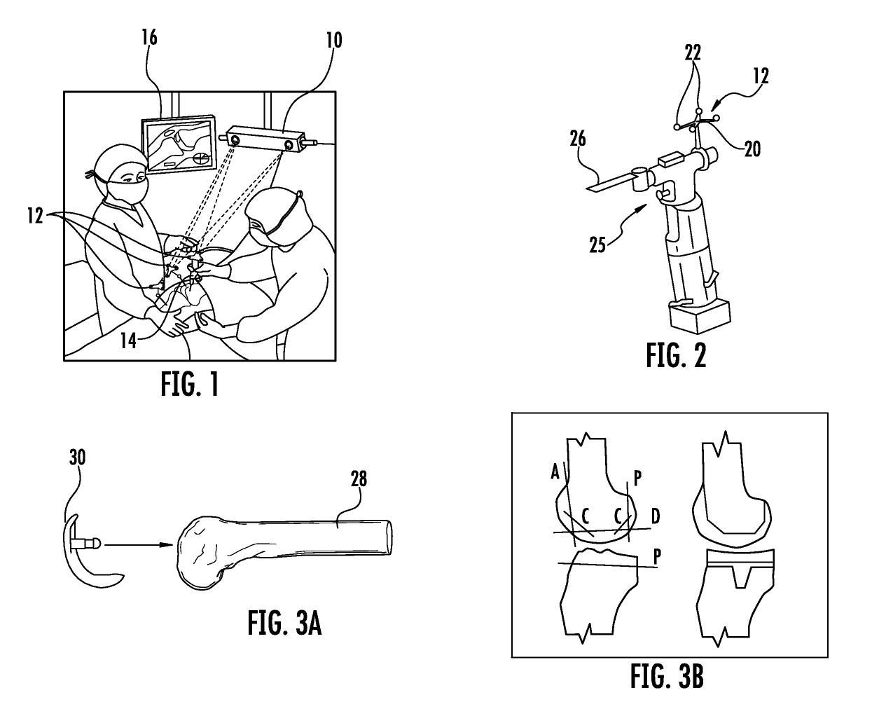

Over the past two decades many surgical navigation systems were developed to increase the accuracy of bone cuts and implant alignment. They rely on position tracking instruments, such as stereoscopic infrared cameras, to localize the position of tracking markers in real time (as discussed herein with reference to FIGS. 1-2). Tracking markers are attached to tools and bones of interest, and the computer compares the position of tools relative to target bones with the surgical plan and communicates this information back to the surgeon. This then allows the surgeon to correct the position of the tool consistent with the plan. Using navigation, the key anatomic landmarks, such as the joint centers, can be localized with higher accuracy and consequently the cut planes can be specified more precisely.

A tracked saw (having surgical navigation) may allow the cutting plane of the saw be known at all times. The surgeon just has to align the saw with the plan and cut the bone without any stabilizers or additional instruments. Although such an arrangement may eliminate the stabilizers and make the cutting faster, it may also produce much rougher surfaces than with conventional instrumentation because, as mentioned before, the blade may flex and the interface that locks the blade to the saw may have significant play in it. Hence, while there is potential to save time and reduce instrumentation, it is desirable to decrease flexure and increase control provided to the surgeon in order to achieve greater accuracy in planar cuts as discussed in more detail herein with reference to various embodiments.

There are several forms of surgical navigation (for example, optical, ultrasound, magnetic, et cetera). Optical navigation is used in surgery to track the location of a rigid body in space. In surgical navigation, an optical tracking system sends tool and bone position information to a computer, where it is converted into clinically relevant data and displayed to the surgeon as guidance. Although the discussion herein is provided in the context of optical navigation, it is understood that the teachings of the present disclosure may be used with other (non-optical) surgical navigation systems as well (for example, systems with non-optical position tracking using, for example, electromagnetic, inertial, or hybrid means).

FIG. 1 illustrates a simplified view of an example surgical navigation setup. An infrared camera 10 and a set of tracker arrays (or, simply, “trackers”) 12 may be used to perform surgical navigation. One of the trackers 12 may be rigidly attached to any object 14 (for example, a surgical drill/hand piece or a surgical saw/stabilizer) that the user wishes to track during the surgical procedure. Each tracker array 12 may consist of a unique configuration of IR reflective markers (for example, markers 22 in FIG. 2).

A camera 10 takes continuous pictures of the workspace during the surgical procedure, and the tracker markers 22 are detected from those pictures. Using the known rigid spatial relationship of the markers 22 on the image frame, the position of the object 14 in a 3D (three dimensional) space can be determined. This location of the object 14 can be continuously output to a computer program that can integrate this location with patient anatomy (such as a CT scan or ultrasound image). The object location relative to the patient anatomy can also be continuously displayed on a display terminal or monitor 16. Thus, the surgeon knows the location of the object 14 relative to the patient. An exemplary optical tracking system that may be used in the context of the present disclosure is the OPTOTRAK CERTUS system (which has 0.15 mm 3D accuracy at over 1000 Hz) or the POLARIS SPECTRA system, both registered trademarks of, and available from, Northern Digital, Inc.

It is understood that multiple objects 14 can be tracked, including rigid patient anatomy such as a bone, in the same workspace with the same camera 10 as illustrated in FIG. 1. As also illustrated in FIG. 1, each object (or part of a patient's anatomy) 14 has its own tracker frame/array and the configuration of the (tracker) markers 22 is unique for each object so as to enable the software (or any other computer processor analyzing image data) to distinguish between objects 14 based on their respective trackers.

In TKR, the volume of the bone removed is substantial and using a bone saw to prepare the bone is appropriate. FIG. 2 illustrates an example sketch of a navigated surgical saw (or “saw”) 25 according to one embodiment. The saw 25 may be used in combination with surgical navigation (discussed herein) to allow the surgeon to make the cuts using a freehand motion. As illustrated in FIG. 2, an example tracker 12 (including a tracker frame 20 and markers 22) may be rigidly attached to the saw 25 to be tracked. The display software can be used to project the geometry of the object 14 being tracked (that is, the surgical saw 25) on the display screen 16 so that a virtual, real-time image of the object 14 and the surrounding anatomy of the patient can be made available to the surgeon to aid in the surgery. A virtual interface may depict the tracked saw 25 in geometrical relationship with the tracked anatomy of a patient. This interface may remain visible to the surgeon (on the display screen 16) during a surgical procedure.

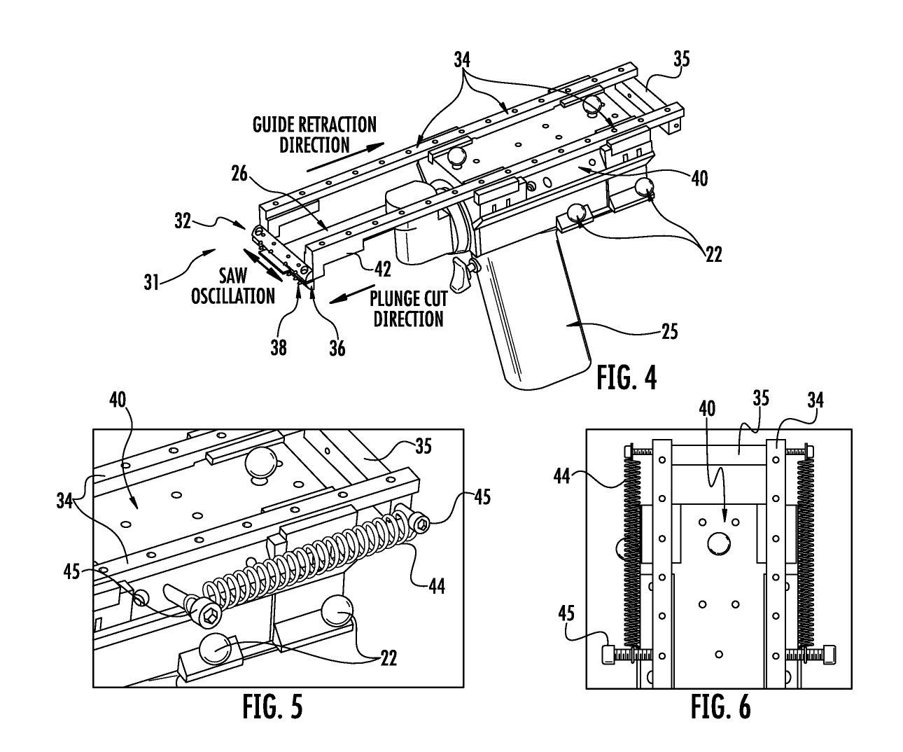

As mentioned, the saw 25 may be supported by navigation visualization (which can include a full 3D visualization of bone models and relative tool positions, as well as a guiding interface for the saw), and may be provided with a blade stabilizer mechanism (discussed herein with reference to FIGS. 4-10) in order to provide accurate and precise cuts. This can lead to improved patient outcomes with lower costs.

In one embodiment, the saw 25 may include a standard sagittal saw (with a blade 26) retrofitted with navigation hardware (that is, the optical trackers 12). The use of optical navigation in conjunction with a standard sagittal saw used for TKR surgery may reduce overall procedure time, may eliminate certain labor-intensive steps and a significant amount of instrumentation needed to carry out the surgery, and may also provide accurate and precise cuts with computer-aided positioning of an implant.

For example in one embodiment, as part of preparing a femur for a knee implant, a series of planar cuts are typically made on the bone using a sagittal saw. These cuts should match the implant surface for an accurate locational fit of implant to bone. FIG. 3A-B shows a femur bone 28 with an exemplary set of planar cuts to enable the bone 28 to receive an implant 30 during the TKR surgery. FIG. 3B shows typical femur and tibia planar cuts (A, C, C, P, P, D) for a TKR surgery. When a sagittal saw is used, it is desirable to make these cuts in the correct orientation with respect to the bone 28 so as to place the implant 30 in the anatomically correct position.

To ensure that the cuts are made at the desired level and orientation, and that the cut surfaces are truly planar, an embodiment provides a blade stabilizer that more rigidly associates the cutting region of the blade relative to the saw body, while allowing the blade to cut freely. FIG. 4 illustrates a sagittal saw 25 mounted with a blade stabilizer 31 according to one embodiment. The blade stabilizer 31 in the embodiment of FIG. 4 comprises a slotted stabilizer 32 and a pair of linear slides 34. The blade stabilizer 31 may further include a slide connector 35 to link the distal ends of the linear slides 34 together as shown in FIGS. 4-5. The slide connector 35 may be either an integral part of the slides 34 or a separate component provided for the linking of the slide ends.

The slotted stabilizer 32 may include a slot 36 and a plurality of bone anchor spikes 38 (FIGS. 8 and 9 illustrate example stabilizer geometry in more detail). It is noted here that, in one embodiment, the blade stabilizer 31 may also include a slide support 40 (that may be either an integral part of the linear slides 34 or a separate component) so as to facilitate level-mounting of the slides 34 onto the top of the navigated saw 25. The blade stabilizer 31 may further include a pair of attachment blocks 42 to facilitate attachment of the slotted stabilizer 32 to the front ends of the slides 34 (that is, those ends which are closest to the saw blade 26) as illustrated in FIG. 4.

In one embodiment, the slotted stabilizer 32 may itself comprise the attachment blocks 42 as part of the stabilizer geometry and, hence, a separate attachment mechanism may not be needed. Instead of providing a separate tracker frame 20 for the navigation markers 22—as is the case in the embodiment of FIG. 2, the slide support 40 and portions of the saw body 25 may be used to provide supporting platforms to receive the navigation markers 22 as is the case in the embodiment of FIG. 4.

Before discussing the operation of the stabilizer-mounted sagittal saw according to one embodiment, it is noted here that the stabilizer 31 (including spikes 38), slides 34, support 40, and attachment blocks 42 may be made of a number of different metals and/or plastics including, for example, anodized aluminum, stainless steel, titanium, ABS (Acrylonitrile Butadiene Styrene), polycarbonate, nylon, et cetera. In one embodiment, the slides 34 may incorporate part nos. 8438k3 (slide block) and 6725k432 (slide rails) available for purchase from McMaster Carr of 200 Aurora Industrial Parkway, Aurora, Ohio 44202-8087, USA. In another embodiment, the slotted stabilizer 32 may be made in a customized configuration from stainless steel and aluminum stock. It is further noted that components or parts having substantially similar or identical functionality may be referred to herein using identical reference numerals for clarity and ease of discussion.

As described herein, the problem of blade flexure (and resulting errors in the tracking system of a navigated surgical saw) may be alleviated by rigidly fixing the cutting region of the saw blade 26 relative to the saw body using the blade stabilizer 31 according to one embodiment. This may be achieved by mounting the slotted stabilizer 32 to the saw 25 via high precision slides 34 using various attachment components illustrated in FIG. 4 and discussed herein. During operation, the saw blade 26 may be inserted in the stabilizer slot 36, which encompasses the blade 26 and acts as a stabilizer to eliminate deflection while cutting. Further, the slot 36 ensures that the saw blade 26 remains in the desired plane during cutting.

In one embodiment, as illustrated in FIG. 5, the stabilizer 31 may be spring loaded using a spring 44 on each side of the slide support 40. FIG. 5 depicts an enlarged view of the spring-containing posterior portion of the stabilizer-mounted saw 25 illustrated in FIG. 4. Two plastic or metallic bolts 45 may used to link the slide connector 35 to a side of the slide support 40 using a spring 44 as illustrated in the embodiment of FIG. 5. Although not visible in FIG. 5, it is observed from the top view provided in FIG. 6 that a second such spring also may be provided on the other side of the support 40, thereby biasing the movement of the slides 34 with the springs 44. In other words, the blade stabilizer 31 may also comprise a pair of springs 44—one spring placed on each side of the support 40—to provide a retractable/compressible mechanism.

Such a retractable mechanism/arrangement results in the spring-loading of the stabilizer 31 so that when a surgeon plunges down into the bone to make a cut, the slotted stabilizer 32 retracts backward (compresses under pressure using the springs 44) and remains on the bone surface, supporting the blade 26 in the desired cut plane for the entire length of the cut and also decreasing blade flexure during the cut (thereby increasing the accuracy of the cut). The springs 44 may allow the slotted stabilizer 32 to return to its original position when the pressure on the slotted stabilizer 32 (that is in immediate contact with the bone surface) is removed (for example, when the surgeon retracts the saw 25 from the bone). The springs 44 may be made of metallic or plastic material and may be selected as per desired tension and extension requirements under operating conditions. In one embodiment, the springs 44 may be standard tension springs. However, in alternative embodiments, a mechanism could be designed that uses compression springs, torsion springs, or pneumatic springs, or some other mechanism that appropriately biases the slotted stabilizer 32 such as pneumatic cylinders, elastomers, et cetera. Thus, FIGS. 5-6 provide an example of a spring-loading arrangement.

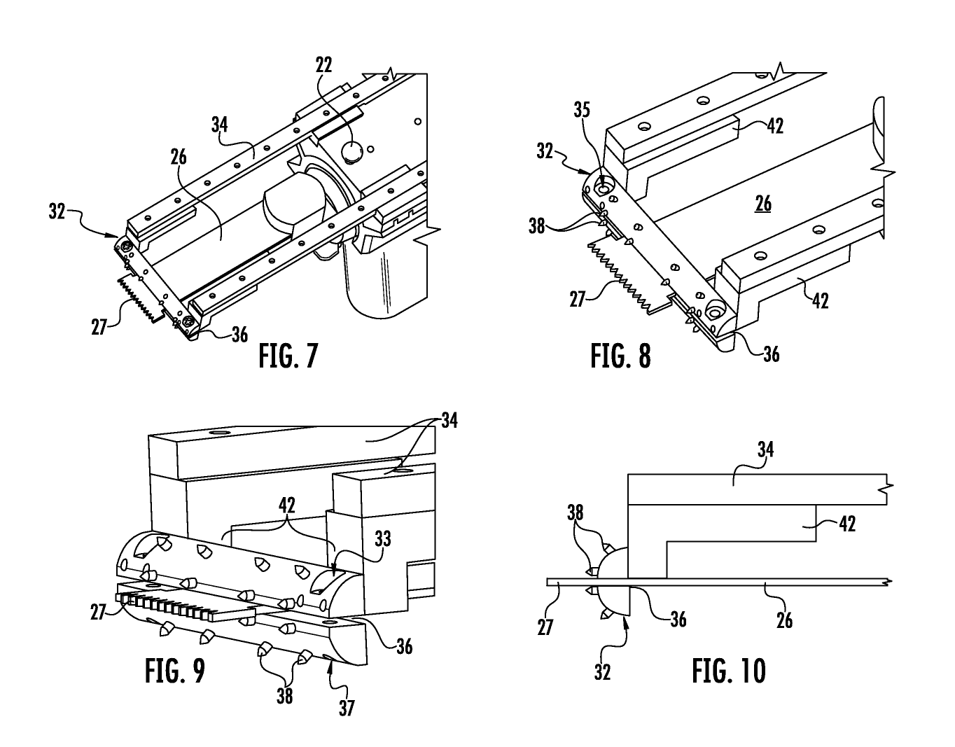

FIG. 7 illustrates a close-up, perspective view of the anterior portion of the stabilizer-mounted saw 25 illustrated in FIG. 4. The saw blade 26 is clearly visible in FIG. 7 with its tip 27 extending from the slot 36 in the stabilizer 32. FIGS. 8 and 9 depict additional close-up views of the slotted stabilizer 32 according to one embodiment. The stabilizer slot 36 and the arrangement of the bone anchor spikes 38 are illustrated in more detail in FIGS. 8 and 9. Other structural elements shown in FIGS. 7-9 are already discussed hereinbefore and, hence, are not further discussed in conjunction with FIGS. 7-9 for the sake of brevity.

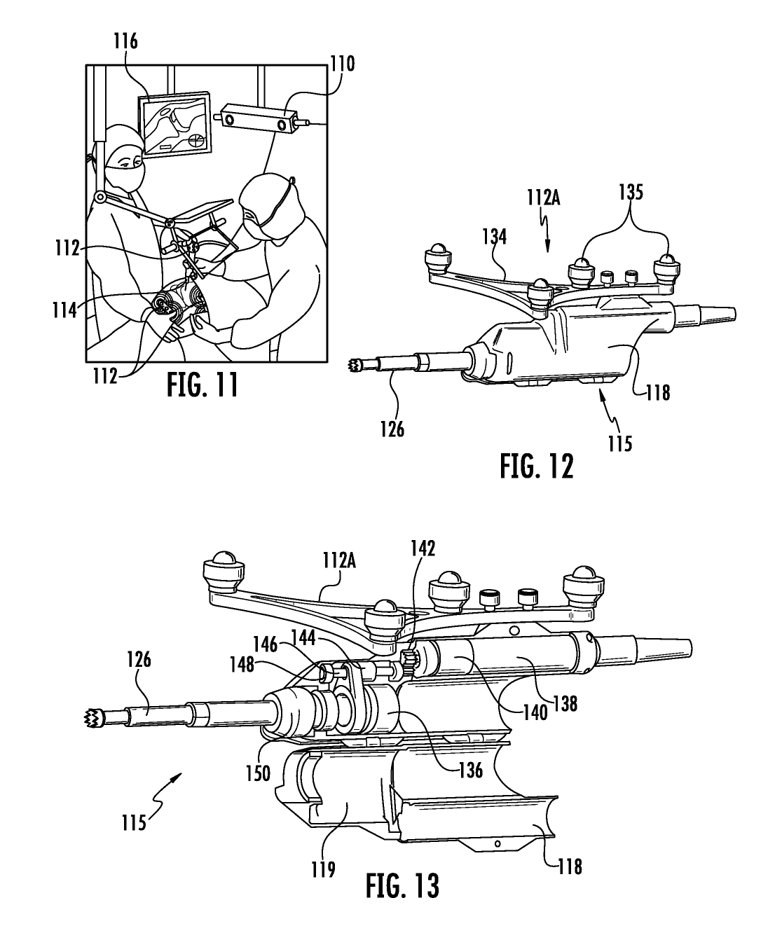

FIG. 10 is a side view corresponding to the perspective view in FIG. 8. The symmetrical arrangement of the spikes 38 around the tip 27 of the saw blade 26 inserted through the slot 36 in the slotted stabilizer 32 according to one embodiment is more clearly visible in the side view of FIG. 10. This arrangement of spikes 38 is but one example. The slotted stabilizer 32 has an upper and a lower portion that may be connected via apertures and screws. As can be seen in FIG. 9, there is a vertical recess 33 in the rounded surface of the upper portion exposing an aperture 35 (refer to FIG. 8) that aligns with another aperture 37 in the lower portion. With this connection, the lower portion may attach to the rest of the stabilizer. It should be noted that other attachments for slotted stabilizer 32 may be utilized.

In order for a sagittal saw to make a precise cut in a desired plane, the plane of the blade 26 should lie coincident with the desired cut plane. Any deviation that the user (surgeon) makes from the correct trajectory (based on the user's inability to keep the saw in the correct and stable position) may be detected by the navigation system via the tracking system (including the navigation markers 22 in FIG. 4). Since the navigation system is tracking the bone to be cut and the saw, any deviation in the trajectory of the saw relative to the bone will be detected by the navigation software and registered as an error. The desired trajectory of the saw may be defined by the surgeon during a pre-planning step where the implant is virtually located on the bone based on the bone anatomy. The software takes the geometry of the implant and lays it on the geometry of the bone. The software can then determine the cuts that need to be made on the bone to place the implant in the correct location. However, it is possible that the surgeon would not be able to keep the trajectory of cut steady and constant if the surgery were to be performed freehand. Hence, during cutting, motion of the blade 26 should be limited to rotation or translation in the desired cut plane, which may be accomplished using the slotted stabilizer 32 as discussed herein.

As described herein, location of the desired cutting planes on the bone can be determined using position tracking and registration of the cutting plane to trackers 12 mounted on the bone. The saw 25 also may be tracked, and therefore the computer can calculate the deviation from the desired plane of the current saw blade 26 position. Simple navigation encodes this difference in a graphical user interface (for example, the display screen 16 illustrated in FIG. 1), and leaves it up to the surgeon to use this feedback to correct for the error in position. However, freehand control of a high-speed cutting device in many degrees of freedom may be difficult, particularly when starting the cut.

The initial contact with bone with an oscillating blade 26 may have a tendency to “kick” off the bone. Maintaining the proper cutter orientation while performing the cutting “plunge” motion may be difficult, particularly because the surgeon must maintain many degrees of freedom at once. The slotted stabilizer 32 addresses this issue by allowing usage of the navigation interface (for example, the display screen 16 illustrated in FIG. 1) to set the start point of the cut, prior to turning on the saw 25.

As described herein, starting the cut freehand using the navigation interface may be difficult due to the vibration and weight of the saw 25. However, the slotted stabilizer 32 includes small spikes 38 that grip the bone with minimal pressure to anchor the initial entry point of the slotted stabilizer 32 (thereby fixating the position of the cutting plane for the saw blade 26). After the initial cut point is set, the user (surgeon) may only need to adjust the angle of cut (that is, the orientation of the cutting plane) and keep the saw 25 aligned to this angle via the (visual) navigation interface while making the cut. This reduces the burden on the surgeon to keep multiple degrees of freedom aligned freehand while cutting with a saw 25 that creates a high level of vibration. In other words, the slotted stabilizer 32 with the spikes 38 provides a stable base on the bone on which the surgeon can adjust cut trajectory using surgical navigation. The stable base provides the physical constraints that limit kickback of the blade 26 and maintain its proper trajectory. Furthermore, as mentioned before, the spring-loading allows the blade stabilizer 31 to be retractable, thereby decreasing flexure of the blade 26 during the cut and, hence, increasing the accuracy of the cut.

It is observed here that the springs 44 are preloaded onto the slides 34, thereby providing the initial force to the spikes 38 on the slotted stabilizer 32 to anchor the slotted stabilizer 32 to the bone. In one embodiment, the spring force is designed to be a balance between having enough force to anchor the slotted stabilizer 32 initially, but without creating excessive resistance during the cut. Thus, in one embodiment, the spring force may be high when starting the cut, so as to provide optimal anchor force in the bone and, hence, to give optimal stabilization to start the cut. Once the cut is started, the spring force may decrease because the cut trajectory becomes more stable when the cut is deeper. In one embodiment, the spring mechanism is such that the spring force is initially high, but it decreases as the cut progresses. In another embodiment, the springs 44 may disengage automatically after the cut has progressed to a certain distance. Such variability in the spring force may allow the surgeon to continue the cut through his/her “feel” of the actual cutting resistance, without any unnecessary distracting forces from the spring mechanism (because the spring force may not be needed or may be needed with lower intensity when the cut has progressed to a certain distance).

It is noted here that although the disclosure is presented with reference to implementation of a blade stabilizer 31 for motion control of a navigated saw 25 used in total knee replacement procedures, it is understood that the principles disclosed herein could be applied to any surgical procedure generally. Thus, the blade stabilizer-based navigated saw 25 according to one embodiment of the present disclosure may be used for any planar bone cut including, for example, in joint arthroplasty, in high tibial osteotomy, in pelvis osteotomy, et cetera. Furthermore, as described herein, different navigation systems may be utilized (for example, not just optical, but ultrasound or magnetic as well).

The blade stabilizer-mounted navigated saw 25 thus gives the surgeon the feel, control, and speed of using a saw 25 freehand while providing an added layer of safety and stability that insures a precise and accurate cut, thereby resulting in more accurate placement of implants. The issue of blade flexure and loose connections may be of secondary importance in case of saws that are not designed to utilize navigation technology. However, when navigation is employed, this issue becomes a hurdle in integrating navigation with this type of a navigated saw 25 (which can be the main tool used for TKR procedures).

The blade stabilizer 31 according to an embodiment thus addresses the problem of blade flexure and the initial starting of a cut while navigating a sagittal saw 25. The spring-loaded retractable blade stabilizer 31 decreases flexure of the blade 26 during a cut (and therefore increases cut accuracy) and the spiked, slotted stabilizer 32 of the stabilizer 31 provides a stable base on the bone on which a user can adjust cut trajectory using surgical navigation. Thus, the stabilizer-mounted saw 25 can facilitate careful preparation of a patient's bone to accept an implant in an anatomically correct and precise location, without significantly adding to the cost or duration of the surgical procedure.

The blade stabilizer 31 may be provided as an augmentation (for example, in the form a kit containing stabilizer components) to a standard sagittal saw used in various surgical procedures. Alternatively, the blade stabilizer 31 may be devised as an integral part of a surgical saw. That is, the stabilizer 31 and the saw 25 may be provided as a single unit. As described herein, the stabilizer 31 enables accurate tracking and placement (on the bone) of the blade of a sagittal saw 25 and, therefore, the trajectory of the cut. This enables the use of various computer-aided surgical tools and methods to be integrated with the saw 25.

An alternative embodiment provides a stabilizer for a surgical drill. Similar to FIG. 1, FIG. 11 illustrates a simplified view of an example surgical navigation setup. An infrared camera 110 and a set of tracker arrays (or, simply, “trackers”) 112 may be used to perform surgical navigation. One of the trackers 112 may be rigidly attached to any object 114 (a surgical drill/hand piece in this example) that the user wishes to track during the surgical procedure. A camera 110, tracker markers 135, and the known rigid spatial relationship of the markers 135 on the image frame allow for determining the position of the object 114 in a 3D space. This location of the object 114 can be continuously output to a computer program that can integrate this location with patient anatomy (such as a CT scan or ultrasound image). The object location relative to the patient anatomy can also be continuously displayed on a display terminal or monitor 116. Thus, the surgeon knows the location of the object 114 relative to the patient.

FIG. 12 illustrates a hand piece assembly 115 that may be included in a tool kit that includes for example a drill, a stabilizer, and other components generally combined to make a surgical tool. These components may be made of a number of different materials such as metals and/or plastics including, for example, anodized aluminum, stainless steel, titanium, ABS (Acrylonitrile Butadiene Styrene), polycarbonate, nylon, et cetera.

Hand piece 115 houses a drill (not shown) and if designed for use with a navigation system may include a tracker 112A assembly, with a tracker frame 134 and markers 135 rigidly attached to the hand piece 115. Markers 135 may be removable and replaceable, if desired. The tracking of the hand piece 115 allows the navigation system to know the position of the effector end of a drill (not illustrated in FIG. 12) when inserted into hand piece 115, as further described herein. The tracker frame 134 may be large in size relative to the actual tracked end effector of the drill (a similar tracker may be mounted on the bone worked upon). Display software may be used to project the geometry of the tracked object (hand piece 115 in this example), on a display screen 116 so that a virtual, real-time image of the hand piece 115 and the surrounding anatomy of the patient bone can be rendered and made available to the surgeon to aid in the surgery. A virtual interface may depict the tracked tool within the hand piece 115 in geometrical relationship with the tracked anatomy of the patient. This interface 116 may remain visible to the surgeon during a surgical procedure.

FIG. 13 illustrates inner constructional details of an example hand piece 115, being hinged to allow opening and access to an inner housing 119 for adjustments, repairs, et cetera. The inner housing 119 of the hand piece 115 may also include a drill moving assembly. In one example, a drill moving assembly includes an actuator 138, a gearhead 140, gears 142, a lead screw nut 144, a lead screw/ball screw 146, and bearings 148 mounted on both ends of the lead screw 146. Accordingly, the hand piece 115 may receive at least a portion of the user-selected drill (not shown) for controlling the generally standard OEM drill and moving the same with respect to hand piece 115. It is noted that drill is positioned within hand piece such that an effector end may protrude through a stabilizer 126 (which encompasses it), as further described herein.

A stabilizer attachment mechanism (support) 150 may further be provided with the hand piece 115 to allow attachment of a cylindrical stabilizer 126 to the attachment mechanism 150 for providing stabilization and shielding, thus allowing for control of the end effector of the drill. As illustrated in FIGS. 12-13, the stabilizer 126 may thus be attached to the attachment mechanism 150 while the inner housing 119 of the hand piece 115 may receive at least a portion of the drill that may be mounted therein. Accordingly, various modular stabilizers 126 may be implemented, as further described herein. Alternatively, it should be noted that a stabilizer 126 may be integrated into hand piece 115 and/or a drill.

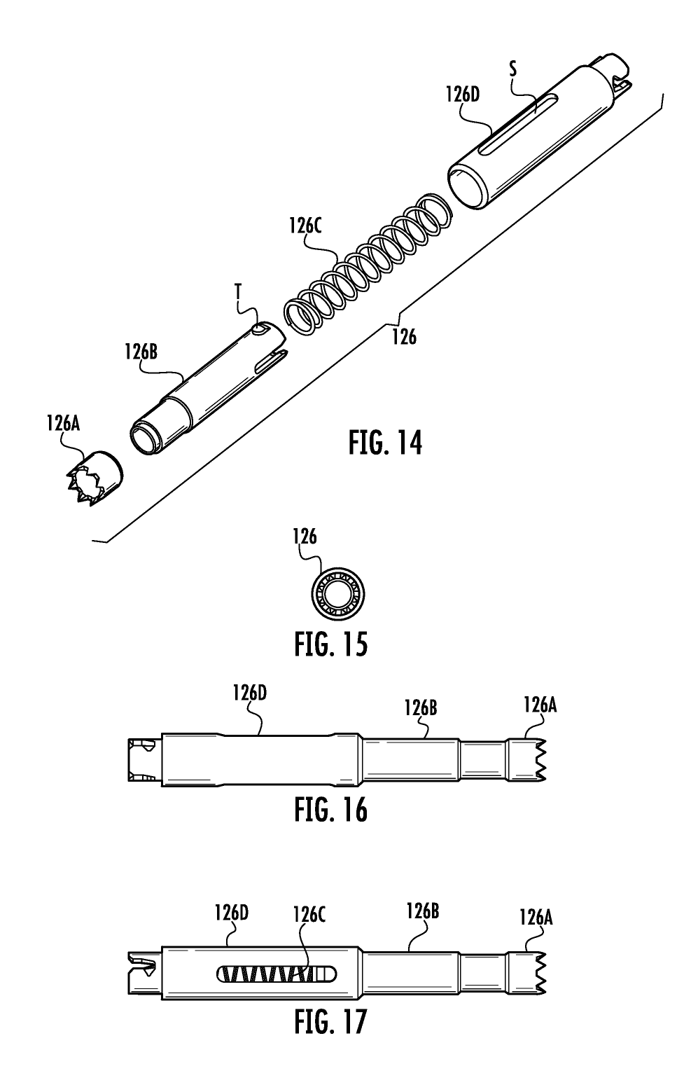

FIG. 14 illustrates an example stabilizer 126. The example stabilizer includes a base component 126D that attaches to attachment mechanism 150 (FIG. 13). The base component 126D interfaces with a plungable component 126B that may be spring loaded by way of inclusion of spring 126C or otherwise arranged such that the stabilizer 126 overall is compressible/retractable, as further described herein. Plungable component 126B may terminate in an insert 126A that includes a plurality of spikes/teeth for gripping an area (such as a contact point on a bone) in order to promote stability of hand piece 115 and thus of a drill contained therein. Thus, the stabilizer 126 provides for stabilizing a drill bit (housed therein) and provides for promoting stable gripping of bone (to prevent drill walking, skiving, et cetera), such as when initiating a cut, similar to that described in connection with a surgical saw. When a surgeon urges the drill contained in hand piece 115 toward the bone, plungable component 126B compresses spring 126C to compress stabilizer 126 overall, allowing an effector end/drill bit to be guided through opening in stabilizer insert 126A, which retracts.

As described in connection with the blade stabilizer for a surgical saw, the spring force of spring 126C may be designed to be a balance between having enough force to anchor the stabilizer initially through gripping of spikes/teeth to bone (or point of contact), but without creating excessive resistance during the drilling motion. Thus, in one embodiment, the spring force may be high when starting the drilling motion, so as to provide optimal anchor force in the bone and, hence, to give optimal stabilization to start. Once started, the spring force may decrease because the drilling trajectory becomes more stable when the drill bit is deeper. In one embodiment, the spring mechanism is such that the spring force is initially high, but it decreases as the drilling progresses. In another embodiment, the springs 44 may disengage automatically after progress to a certain distance/depth has been made. Such variability in the spring force may allow the surgeon to continue the drilling through his/her “feel” of the actual resistance, without any unnecessary distracting forces from the spring mechanism (because the spring force may not be needed or may be needed with lower intensity when the motion has progressed to a certain distance).

An end view (of insert 126A end of stabilizer 126 of FIG. 14) is illustrated in FIG. 15. As shown in FIG. 15, the stabilizer 126 is hollow, permitting a drill bit to be housed therein and, when stabilizer 126 is compressed, as described herein, protrude through.

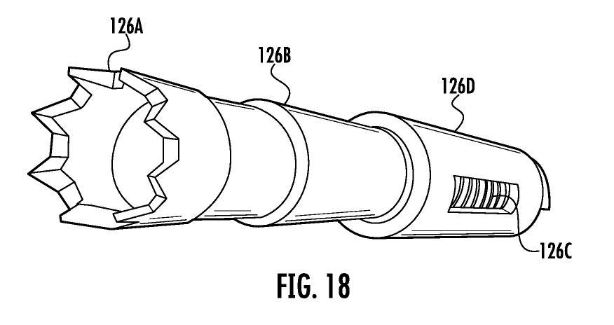

FIGS. 16-17 illustrate side views of stabilizers. In one example, base component 126D is illustrated with slot S (illustrated in FIG. 14) such that tab T of component 126B is positioned therein (when assembled), limiting telescopic travel. Again, as plungable component 126B moves toward base component 126D, spring 126C is compressed and allows plungable component to reveal a drill bit housed therein. As can be appreciated from the example view illustrated in FIG. 18, spikes/teeth of insert 126A provide gripping means for fixing the stabilizer in a fixed position such that when plungable component 126B recesses into base component 126D, compressing spring 126C, an effector end/drill bit (not shown) housed within the stabilizer 126 may be stably urged into an object such as a bone.

Thus, embodiments provide various stabilization arrangements for surgical tools. An embodiment provides a stabilization arrangement for a surgical saw. Another embodiment provides a stabilization arrangement for a surgical drill.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Although illustrated example embodiments have been described herein with reference to the accompanying drawings, it is to be understood that embodiments are not limited to those precise example embodiments, and that various other changes and modifications may be affected therein by one skilled in the art without departing from the scope or spirit of the disclosure.

What is claimed is:

1. A surgical tool stabilizer, comprising:
a support configured to engage at least a portion of a surgical tool and configured to receive at least a portion of a tracking system; and
a retractable stabilizer configured to surround at least a portion of an end effector of said surgical tool.

2. The surgical tool stabilizer of claim 1, wherein said end effector of a surgical tool is a surgical saw blade.

3. The surgical tool stabilizer of claim 2, wherein said retractable stabilizer further comprises:
a guided slot portion having a slot defined therein;
wherein said slot is configured to enclose and stabilize said surgical saw blade.

4. The surgical tool stabilizer of claim 3, wherein said guided slot portion comprises a plurality of spikes.

5. The surgical tool stabilizer of claim 3, further comprising a pair of linear slides;
wherein said guided slot portion is disposed between said pair of linear slides; and
wherein said pair of linear slides comprise a spring mechanism imparting retractability to said retractable stabilizer.

6. The surgical tool stabilizer of claim 5, further comprising one or more markers;
wherein said one or more markers comprise surgical navigation markers.

7. The surgical tool stabilizer of claim 1, wherein said end effector of a surgical tool is a drill bit of a surgical drill.

8. The surgical tool stabilizer of claim 7, wherein said retractable stabilizer further comprises:
a cannulated, plungable drill stabilizer.

9. The surgical tool stabilizer of claim 8, wherein said cannulated, plungable drill stabilizer is conical in shape and terminates in a plurality of spikes.

10. The surgical tool stabilizer of claim 9, further comprising one or more markers;
wherein said one or more markers comprise surgical navigation markers.

11. A surgical tool stabilizer, comprising:
a support configured to engage at least a portion of a surgical saw and configured to receive at least a portion of a tracking system; and
a retractable stabilizer configured to surround at least a portion of an end effector of said surgical saw;
wherein said retractable stabilizer further comprises:
a guided slot portion having a slot defined therein;
wherein said slot is configured to enclose and stabilize said end effector.

12. The surgical tool stabilizer of claim 11, wherein said guided slot portion comprises a plurality of spikes.

13. The surgical tool stabilizer of claim 12, further comprising a pair of linear slides;
wherein said guided slot portion is disposed between said pair of linear slides; and
wherein said pair of linear slides comprise a spring mechanism imparting retractability to said retractable stabilizer.

14. The surgical tool stabilizer of claim 13, further comprising one or more markers;
wherein said one or more markers comprise surgical navigation markers.

15. A surgical tool stabilizer, comprising:
a support configured to engage at least a portion of a surgical drill and configured to receive at least a portion of a tracking system; and
a retractable stabilizer configured to surround at least a portion of an end effector of said surgical drill;
wherein said retractable stabilizer further comprises a cannulated, plungable drill stabilizer.

16. The surgical tool stabilizer of claim 15, wherein said cannulated, plungable drill stabilizer is conical in shape and terminates in a plurality of spikes.

17. The surgical tool stabilizer of claim 16, further comprising one or more markers;
wherein said one or more markers comprise surgical navigation markers.

18. The surgical tool stabilizer of claim 17, wherein said a cannulated, plungable drill stabilizer comprises three portions.

19. The surgical tool stabilizer of claim 18, wherein said three portions comprise a base portion, a plungable portion, and an insert portion comprising the plurality of spikes.

20. The surgical tool stabilizer of claim 19, further comprising one or more springs.
